(19) United States
(12) Patent Application Publication
(10) Pub. No.: US 2006/0257880 A1
(43) Pub. Date: Nov. 16, 2006

(54) Gastric and prostate cancer associated antigens

(75) Inventors: Robert Charles Rees, Bradway (GB); Geng Li, Beeston (GB); Amanda Miles, Heanor (GB)

Correspondence Address: BARNES & THORNBURG LLP, 11 SOUTH MERIDIAN, INDIANAPOLIS, IN, 46204, US

(73) Assignee: THE NOTTINGHAM TRENT UNIVERSITY

(21) Appl. No.: 10/569,572

(22) PCT Filed: Aug. 27, 2004
(86) PCT No.: PCT/GB04/03650

§ 371 Date: May 25, 2006

(30) Foreign Application Priority Data
Aug. 29, 2003 (GB) 0320305.6
Apr. 2, 2004 (GB) 0407586.7

(51) Int. Cl. C12Q 1/68; G01N 33/574; C07H 21/04; C07K 14/82; C07K 16/30

(52) U.S. Cl. 435/6; 435/7.23; 530/350; 530/388.8; 536/23.5

(57) ABSTRACT

The invention relates to an antigen associated with cancers such as prostate and gastric cancers, known as T21. Nucleic acid sequences and polypeptide sequences encoding the antigen are provided, as are uses of such sequences.

The invention relates to isolated nucleic acid sequences which are expressed in cancers, including leukaemias, lymphoma and especially gastrointestinal and prostate cancers, to their protein products and to the use of the nucleic acid and protein products for the identification and treatment of cancers.

Cancers of the intestinal tract, such as gastric carcinomas and colorectal cancers, account for up to 15% of cancer-related deaths in the United States, and have low survival rates. Such cancers are often asymptomatic, the patient only becoming aware of them when the cancers have progressed too far to be successfully treated. There is therefore a need to identify new diagnostic tools and methods for treating such cancers.

The prostate gland is an accessory sex gland in males which is wrapped around the urethra as this tube leaves the bladder. The gland secretes an alkaline fluid during ejaculation. Cancer of the prostate gland is very serious and represents the second leading cause of death from cancer in men.

Two specific proteins are known to be made in very high concentrations in prostate cancer cells. These are prostatic acid phosphatase (PAP) and prostate specific antigen (PSA). These proteins have been characterised and have been used to follow response to therapy. However, it has been difficult to correlate the presence of these two proteins to the presence of cancer.

Accordingly, there is a need to identify new genes and proteins which are associated with the presence of prostate cancer and other cancers.

Identification of immunogenic proteins in cancer is essential for the development of immunotherapeutic strategies where adoptive immunity is directed towards MHC Class I- and Class II-associated peptides (Mian, et al., in Cancer Immunology (2001), page 1-26 Eds: R. Adrian Robins and Robert C. Rees, Immunology and Medicine Series, Kluwer Academic Publishers). Many antigens are implicated in aetiology and progression of cancer, and are associated with epigenetic events. Pre-clinical and clinical studies infer that vaccination and targeting MHC-associated peptide antigens promotes tumour rejection (Ali S. A., et al, J Immunol. (2002), Vol. 168(7), pages 3512-19 and Rees R. C., et al., Cancer Immunol. Immunother (2002), Vol 51(1), pages 58-61).

The inventors have used a technique known as SEREX (Serological Analysis of Recombinant cDNA Expression Libraries) to identify genes which are over-expressed in cancer tissue. This technique was published by Sahin et al (PNAS (USA), 1995, Vol. 92, pages 11810-11813). SEREX normally uses total RNA isolated from tumour biopsies from which poly(A)⁺ RNA is then isolated. cDNA is then produced using an oligo (dT) primer. The cDNA fragments produced are then cloned into a suitable expression vector, such as a bacteriophage and cloned into a suitable host, such as Escherichia coli. The clones produced are screened with high-titer IgG antibodies in autologous patient serum, to identify antigens associated with the tumour.

Several SEREX-defined antigens have provided attractive candidates for the construction of cancer vaccines, for example NY-ESO-1 from testis (Chen Y. T., et al. (1997), Vol. 4, page 1914; Stockert E., et al., J. Exp. Med. (1998), Vol. 187, page 1349; Jager E., et al. PNAS (2000), Vol. 97, page 12198; and Jager E., et al., PNAS (2000), Vol. 97, page 4760). Mutated p53 (Scanlan M. J., et al., Int. J. Cancer (1998), Vol. 76, page 652), putative tumour suppresser ING 1 (Jager D. et al., Cancer Res. (1999), Vol. 59, page 6416) and adhesion molecule galectin 9 (Tureci O., et al., J. Biol. Chem. (1997), Vol. 272, page 6416), for example, have been detected by SEREX, showing that the analysis of autoantibodies can identify genes involved in cancer aetiology and identify diagnostic markers or indicators of disease progression.

The inventors unexpectedly realised that some cancers express antigens that are also found in normal testes tissues. They therefore took the unusual step of adapting the SEREX technique to screen a normal testicular tissue cDNA library against serum from pooled allogeneic prostate cancer patients.

The testes cDNA library was screened using pooled allogeneic prostate cancer patients' sera. Seven reactive clones were purified, in vivo excised, and converted to plasmid forms. cDNA inserts were analysed using restriction mapping and cDNA sequencing. Comparison to the Genbank non-redundant and expressed sequence tag (EST) databases revealed that these 7 clones represented 6 distinct genes, 5 previously unknown genes and 1 known gene. The first (designated T21) of these to be fully sequenced is described here; the following results are consistent with T21 being a cancer-testis (CT) associated gene, the predicted protein of which are likely to act as a new target antigen for immunotherapy.

It was unexpectedly found that T21 is highly expressed in malignant tissues, especially prostate and gastric cancers. Analysis of the DNA encoding the initially identified T21 antigen revealed that the sequence was truncated at both the 3′ and 5′ ends. Further investigation, using Rapid Amplification of the cDNA Ends (RACE) experiments, was required in order to identify the full-length DNA sequence which encodes T21.

By searching the GenBank database the inventors found T21 exhibited restricted homology to a previously published sequence representing a homo sapiens MAb 3H11 antigen AAK01919 (GenBank accession number AF317887). This is described in more detail in Biochem. Biophys. Res. Common, Vol. 280(1), pages 99-103 (2001). At the amino acid level, in positions 1-77 only, T21 shows 48% sequence identity to MAb 3H11 antigen, which is known to be a tumour associated antigen with highly restricted normal tissue expression and increased levels of expression observed in gastric cancer.

Motif analysis of the amino acid sequence using PROSITE, PSORT and Pfam search programs identified:

(i) N-glycosylation sites at amino acid positions 158-161, 430-433 and 466-469;

(ii) cAMP and cGMP-dependent protein kinase phosphorylation sites at positions 314-317 and 487-490;

(iii) Casein kinase II phosphorylation sites at positions 6-9, 34-37, 152-155, 209-212, 234-237, 258-261, 269-272, 332-335, 388-391, 390-393, 421-424, 432-435, 468-471, 478-481 and 490-493;

(iv) N-myristoylation sites at positions 99-104, 123-128, 198-203 and 465-470;

(v) leucine zipper patterns at positions 214-235 and 319-340;

(vi) bZIP sites 60-97, 130-165, 424-453;

(vii) k-box sites 33-48, 511-533; and

(viii) SPAN-X domain 293-315.

PSORT analysis predicted with 60% confidence the nuclear localisation of T21 protein. bZIP sites (DNA-binding site followed by a leucine zipper motif) at positions 60-97, 130-165 and 424-453 suggests that this protein may function as a transcription factor. In addition, k-box regions at positions 33-48 and 511-533 are commonly associated with SRF-type transcription factors.

Protein kinase C phosphorylation sites at positions 139-141, 152-154, 258-260, 262-264, 269-271, 313-315, 403-405, 432-434, 486-488 and 516-518 suggests that this clone might be involved in signalling pathways. B-cell receptor-associated protein 31-like domain, Bap31, at position 433-445 suggests a possible role in the induction of apoptosis. Bap31 is a polytopic integral protein of the endoplasmic reticulum membrane and a substrate of caspase-8. Bap31is cleaved within its cytosolic domain, generating pro-apoptotic p20 Bap31.

Interestingly, the protein has a SPAN-X domain at position 293-315 and this family contains human sperm proteins associated with the nucleus and mapped to chromosome X.

SPAN-X proteins are cancer-testis antigens that are highly insoluble, acidic and polymorphic.

The protein bound by MAb 3H11 has an identical region to T21 of only 28 consecutive amino acids out of 534 amino acids in the peptide sequence of the T21 protein (i.e. residues 49 to 66 of T21). Both T21 and MAb 3H11 are encoded by genes located on chromosome 12. T21 is a previously unknown antigen.

By “the protein bound by MAb 3H11” it is meant that the protein is the protein identified in the Chen and Shou article (Chen, D. and Shou, C., Biochem. Biophys. Res. Commun., Vol. 280 (2001), pages 99-102) and listed as Genbank Acc. No. AF317887.

The invention provides an isolated mammalian nucleic acid molecule selected from the group consisting of:

(a) Nucleic acid molecules encoding a polypeptide comprising the amino acid sequence depicted from SEQ. ID. 3 amino acid number 77 to amino acid 535

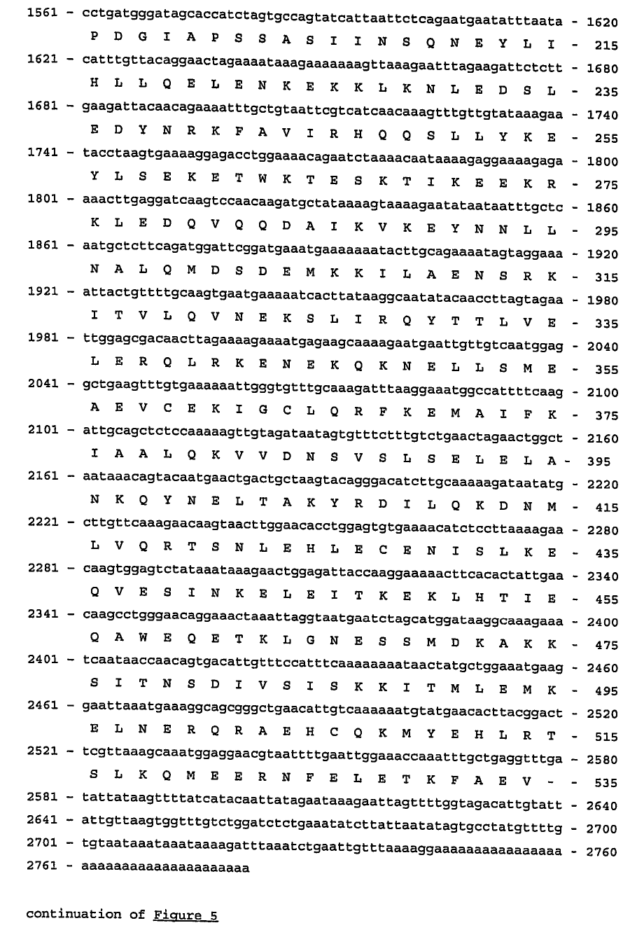

(b) Nucleic acid molecules comprising the nucleotide sequence depicted in SEQ. ID. 1, SEQ. ID. 2 or FIG. 5.

(c) Nucleic acid molecules, the complementary strand of which specifically hybridises to a nucleic acid molecule in (a) or (b).

(d) Nucleic acid molecules the sequence of which differs from the sequence of the nucleic acid molecule of (c) due to the degeneracy of the genetic code.

Preferably the nucleic acid molecule encodes T21. T21 is expressed in higher than normal concentrations in normal testes tissue, compared to one or more of normal lung, liver, heart, brain, trachea, adrenal gland, endometrium, colon, breast, PBMC, tonsil, small intestine, vagina, muscle, placenta, ovary, and/or prostate.

Preferably the antigen is expressed in higher concentrations of normal testicular tissue compared with the normal tissues listed above.

Preferably the nucleic acid molecule encodes a polypeptide which is capable of acting as a transcription factor. That is, it is capable of binding a DNA molecule and regulating the transcription of a region of that DNA molecule by an RNA polymerase. Most preferably the polypeptide has one or more of the following features:

(i) N-glycosylation site

(ii) cAMP dependent protein kinase phosphorylation site

(iii) cGMP dependent protein kinase phosphorylation site

(iv) casein kinase II phosphorylation site

(iv) N-myristoylation site

(v) leucine zipper pattern

(vi) bZIP site

(vii) k-box site

(viii) SPAN-X domain

Nucleic acid molecules having at least 60%, 70%, 75%, 80%, 85%, 90%, 95%, 98%, 99% homology to the nucleic acid molecules are also provided. Preferably these express proteins which are expressed in higher concentrations in cancerous tissue than the equivalent normal tissue. That is they are higher in e.g. prostate cancer or gastric cancers than normal, non-cancerous, prostate or normal non-cancerous gastric tissue. The proteins are preferably expressed in higher concentrations in testes than in e.g. one or more of normal lung, liver, heart, brain, trachea, adrenal gland, endometrium, colon, breast, PBMC, tonsil, small intestine, vagina, muscle, placenta, ovary, and/or prostate. Preferably this is at least 2, most preferably at least 5 times higher concentrations than normal tissue.

The nucleic acid molecules of the invention may be DNA, cDNA or RNA. In RNA molecules “T” (Thymine) residues may be replaced by “U” (Uridine) residues.

Preferably, the isolated mammalian nucleic acid molecule is an isolated human nucleic acid molecule.

The invention further provides nucleic acid molecules comprising at least 15 nucleotides capable of specifically hybridising to a sequence included within the sequence of a nucleic acid molecule according to the first aspect of the invention but not to the protein bound by MAb 3H11. The hybridising nucleic acid molecule may either be DNA or RNA. Preferably the molecule is at least 90%, at least 92%, at least 94%, at least 96%, at least 98%, at least 99%, homologous to the nucleic acid molecule according to the first aspect of the invention. This may be determined by techniques known in the art.

That is, preferably the nucleic acid molecule does not specifically hybridise to a part of SEQ. ID. No. 2 from residue 115 to 198.

The term “specifically hybridising” is intended to mean that the nucleic acid molecule can hybridise to nucleic acid molecules according to the invention under conditions of high stringency. Typical conditions for high stringency include 0.1×SET, 0.1% SDS at 68° C. for 20 minutes.

The invention also encompasses variant nucleic acid molecules such as DNAs and cDNAs which differ from the sequences identified above, but encode the same amino acid sequences as the isolated mammalian nucleic acid molecules, by virtue of redundancy in the genetic code.

| | U | | | C | | | A | | | G | | | |
|---|---|---|---|---|---|---|---|---|---|---|---|---|---|
| U | UUU | | | UCU | | | UAU | | | UGU | | | U |
| | | {close oversize bracket} | Phe | | | | | {close oversize bracket} | Tyr | | {close oversize bracket} | Cys | |
| | UUC | | | UCC | | | UAC | | | UGC | | | C |
| | UUA | | | UCA | {close oversize bracket} | Ser | UAA* | | Stop | UGA* | | Stop | A |
| | | {close oversize bracket} | Leu | | | | | | | | | | |
| | UUG | | | UCG | | | UAG* | | Stop | UGG | | Trp | G |
| C | CUU | | | CCU | | | CAU | | | CGU | | | U |
| | | | | | | | | {close oversize bracket} | His | | | | |
| | CUC | | | CCC | | | CAC | | | CGC | | | C |
| | CUA | {close oversize bracket} | Leu | CCA | {close oversize bracket} | Pro | CAA | | | CGA | {close oversize bracket} | Arg | A |
| | | | | | | | | {close oversize bracket} | Gln | | | | |
| | CUG | | | CCG | | | CAG | | | CGG | | | G |
| A | AUU | | | ACU | | | AAU | | | AGU | | | U |
| | | | | | | | | {close oversize bracket} | Asn | | {close oversize bracket} | Ser | |
| | AUC | {close oversize bracket} | Ile | ACC | | | AAC | | | AGC | | | C |
| | AUA | | | ACA | {close oversize bracket} | Thr | AAA | | | AGA | | | A |
| | | | | | | | | {close oversize bracket} | Lys | | {close oversize bracket} | Arg | |
| | AUG** | | Met | ACG | | | AAG | | | AGG | | | G |
| G | GUU | | | GCU | | | GAU | | | GGU | | | U |
| | | | | | | | | {close oversize bracket} | Asp | | | | |
| | GUC | | | GCC | | | GAC | | | GGC | | | C |
| | GUA | {close oversize bracket} | Val | GCA | {close oversize bracket} | Ala | GAA | | | GGA | {close oversize bracket} | Gly | A |
| | | | | | | | | {close oversize bracket} | Glu | | | | |
| | GUG** | | | GCG | | | GAG | | | GGG | | | G |

*Chain-terminating, or “nonsense” codons.
**Also used to specify the initiator formyl-Met-tRNAMet. The Val triplet GUG is therefore “ambiguous” in that it codes both valine and methionine.

The Genetic Code Showing mRNA Triplets and the Amino Acids for Which They Code

The invention also includes within its scope vectors comprising a nucleic acid according to the invention. Such vectors include bacteriophages, phagemids, cosmids and plasmids. Preferably the vectors comprise suitable regulatory sequences, such as promoters and termination sequences which enable the nucleic acid to be expressed upon insertion into a suitable host. Accordingly, the invention also includes hosts comprising such a vector. Preferably the host is E. coli.

A second aspect of the invention provides an isolated polypeptide obtainable from a nucleic acid sequence according to the invention. As indicated above, the genetic code for translating a nucleic acid sequence into an amino acid sequence is well known.

Preferably the sequence comprises the sequence shown in SEQ. ID. 3.

The invention further provides polypeptide analogues, fragments or derivatives of antigenic polypeptides which differ from naturally-occurring forms in terms of the identity of location of one or more amino acid residues (deletion analogues containing less than all of the residues specified for the protein, substitution analogues wherein one or more residues specified are replaced by other residues in addition analogues wherein one or more amino acid residues are added to a terminal or medial portion of the polypeptides) and which share some or all properties of the naturally-occurring forms. Preferably such polypeptides comprise between 1 and 20, preferably 1 and 10 amino acid deletions or substitutions. These do not contain a sequence of 5 or more, preferably 6, 7, 8, 9, 10, 15, 20 or more consecutive amino acid residues from residue 39 to 77 of SEQ. ID. No. 3.

Preferably, the fragment is a sequence selected from:

(i)

| Peptide name | Sequence |
|---|---|
| T21:765 | YLIHLLQEL |
| T21:656 | LIIPSLERL |
| T21:660 | SLERLVNAI |
| T21:607 | VIAKFQNKL |

(ii) a derivative of one of the peptide sequences (i) having one or more amino acid deletions, additions or modifications, and

(iii) a fragment of the fragment (i) or derivative (ii),

wherein the fragment or derivative has HLA-A2 restricted activity.

Isolated nucleic acid molecules encoding such fragments are also included within the scope of the invention.

Preferably the fragment is a peptide.

Most preferably, the fragment comprises the sequence shown as T21:765.

The term peptide preferably means 30 or less, less than 25, 24, 23, 22, 21, 20, 19, 18, 17, 16, 15, 14, 13, 12, 11, 10, 9, 8, 7, 6 or 5 amino acid residues covalently joined to form the polypeptide.

Preferably 1, 2, 3, 4 or 5 amino acids are substituted, added or deleted. The production of such derivatives is achieved by methods known in the art. Preferably such derivatives have improved HLA-A2 restricted activity.

Amino acids are grouped into amino acids having similar properties, e.g.:

Hydrophobic: valine, leucine, isoleucine, methionine, proline

Aromatic: phenylalanine, tyrosine, tryptophan

Basic: lysine, arginine, histidine

Acidic: aspartate, glutamate

Amide: asparagine, glutamine

Nucleophilic: serine, threonine, cysteine

Small: glycine, alanine

Preferably, an amino acid of one group (e.g. basic amino acid) may be substituted for another amino acid from that group.

The “activity” of a peptide is a semi-quantitative measure of its immunogenic potency. For HLA-A2 bound peptide, activity is preferably measured by the extent of lysis by cytotoxic T-cells of target cells displaying the MHC Class I peptide complexes. A peptide is usually considered to be immunogenic if it mediates killing of at least 15% of the cells that display it.

More preferably the term “HLA-A2 restricted activity” means that the polypeptide has activity selected from one or more of:

(i) HLA-A2 binding, especially to HLA-A2*0201. Preferably such binding is with high binding affinity,

(ii) Produces cytotoxicity in splenocytes derived from polypeptide immunised mice. This is described in more detail in the examples.

(iii) The polypeptide produces increase IFN-γ production in splenocytes from polypeptide immunised mice. This is compared with non-immunised mice.

Binding activity may be determined by techniques known in the art.

Preferably the methods of assaying such activity is as shown in the Materials and Methods section.

Preferably the polypeptide is at least 80%, 85%, 90%, 95%, 96%, 97%, 98% or 99% identical to the sequences of the invention. This can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The nucleic acids and polypeptide of the invention are preferably identifiable using the SEREX method. However, alternative methods, known in the art, may be used to identify nucleic acids and polypeptides of the invention. These include differential display PCR (DD-PCR), representational difference analysis (RDA) and suppression subtracted hybridisation (SSH).

The nucleic acid molecules encoding T21 according to the invention and the polypeptides which they encode are detectable by SEREX (discussed below). The technique uses serum antibodies from cancer patients to identify the molecules. It is therefore the case that the gene products identified by SEREX are able to evoke an immune response in a patient and may be considered as antigens suitable for potentiating further immune reactivity if used as a vaccine or in cell based therapies e.g. T-cell adaptive therapy.

The third aspect of the invention provides the use of nucleic acids or polypeptides according to the invention, to detect or monitor cancers, preferably gastro-intestinal cancers, such as gastric cancer, or prostate cancer.

The use of a nucleic acid molecule hybridisable under high stringency conditions, a nucleic acid according to the first aspect of the invention to detect or monitor cancers, e.g. gastro-intestinal cancers, such as gastric cancer or colorectal cancer, or prostate cancer, is also encompassed. Such molecules may be used as probes, e.g. using PCR.

The expression of genes, and detection of their polypeptide products may be used to monitor disease progression during therapy or as a prognostic indicator of the initial disease status of the patient.

There are a number of techniques which may be used to detect the presence of a gene, including the use of Northern blot and reverse transcription polymerase chain reaction (RT-PCR) which may be used on tissue or whole blood samples to detect the presence of cancer associated genes. For polypeptide sequences in-situ staining techniques or enzyme linked ELISA assays or radio-immune assays may be used. RT-PCR based techniques would result in the amplification of messenger RNA of the gene of interest (Sambrook, Fritsch and Maniatis, Molecular Cloning, A Laboratory Manual, 2^{nd} Edition). ELISA based assays necessitate the use of antibodies raised against the protein or peptide sequence and may be used for the detection of antigen in tissue or serum samples (McIntyre C. A., Rees R. C. et. al., Europ. J. Cancer 28, 58-631 (1990)). In-situ detection of antigen in tissue sections also rely on the use of antibodies, for example, immuno peroxidase staining or alkaline phosphatase staining (Goepel, J. R., Rees, R. C. et.al., Brit. J. Cancer 64, 880-883 (1991)) to demonstrate expression. Similarly radio-immune assays may be developed whereby antibody conjugated to a radioactive isotope such as I¹²⁵ is used to detect antigen in the blood.

Blood or tissue samples may be assayed for elevated concentrations of the nucleic acid molecules or polypeptides.

Preferably elevated levels of the molecules in tissues that are not normal testes is indicative of the presence of cancerous tissues.

Methods of producing antibodies which are specific to the polypeptides of the invention, for example, by the method of Kohler & Milstein to produce monoclonal antibodies, are well known. A further aspect of the invention provides an antibody which specifically binds to a polypeptide according to the invention, but not to the protein bound by MAb 3H11.

Kits for detecting or monitoring cancer, such as gastro-intestinal cancers, including gastric cancer and/or colorectal cancer, or prostate cancer, using polypeptides, fragments, nucleic acids or antibodies according to the invention are also provided. Such kits may additionally contain instructions and reagents to carry out the detection or monitoring.

The fourth aspect of the invention provides for the use of nucleic acid molecules according to the first aspect of the invention or polypeptide molecules according to the second aspect of the invention in the prophylaxis or treatment of cancer, or pharmaceutically effective fragments thereof. By pharmaceutically effective fragment, the inventors mean a fragment of the molecule which still retains the ability to be a prophylactant or to treat cancer. The cancer may be a gastro-intestinal cancer, such as gastric cancer or colorectal cancer.

The molecules are preferably administered in a pharmaceutically amount. Preferably the dose is between 1 μg/kg. to 10 mg/kg.

The nucleic acid molecules may be used to form DNA-based vaccines. From the published literature it is apparent that the development of protein, peptide and DNA based vaccines can promote anti-tumour immune responses. In pre-clinical studies, such vaccines effectively induce a delayed type hypersensitivity response (DTH), cytotoxic T-lymphocyte activity (CTL) effective in causing the destruction (death by lysis or apoptosis) of the cancer cell and the induction of protective or therapeutic immunity. In clinical trials peptide-based vaccines have been shown to promote these immune responses in patients and in some instances cause the regression of secondary malignant disease. Antigens expressed in prostate cancer (or other types of cancers) but not in normal tissue (or only weakly expressed in normal tissue compared to cancer tissue) will allow us to assess their efficacy in the treatment of cancer by immunotherapy. Polypeptides derived from the tumour antigen may be administered with or without immunological adjuvant to promote T-cell responses and induce prophylactic and therapeutic immunity. DNA-based vaccines preferably consist of part or all of the genetic sequence of the tumour antigen inserted into an appropriate expression vector which when injected (for example via the intramuscular, subcutaneous or intradermal route) cause the production of protein and subsequently activate the immune system. An alternative approach to therapy is to use antigen presenting cells (for example, dendritic cells, DC's) either mixed with or pulsed with protein or peptides from the tumour antigen, or transfect DC's with the expression plasmid (preferably inserted into a viral vector which would infect cells and deliver the gene into the cell) allowing the expression of protein and the presentation of appropriate peptide sequences to T-lymphocytes or adaptive cellular therapy using, e.g., T-cells responsive to T21 peptides or T21 protein. A DNA based vaccine is demonstrated in, for example, Tompston S. A., et al. (J. Immunol. (1998) Vol. 160, pages 1717-1723).

Accordingly, the invention provides a nucleic acid molecule according to the invention in combination with a pharmaceutically-acceptable carrier.

A further aspect of the invention provides a method of prophylaxis or treatment of a cancer such as a gastro-intestinal cancer, or prostate cancer, comprising the administration to a patient of a nucleic acid molecule according to the invention.

Such polypeptides may be bound to a carrier molecule such as tetanus toxoid to make the polypeptide immunogenic. Such constructs are also within the scope of the invention

The polypeptide molecules according to the invention may be used to produce vaccines to vaccinate against a cancer, such as a gastro-intestinal cancer or prostate cancer.

Accordingly, the invention provides a polypeptide according to the invention in combination with a pharmaceutically acceptable carrier.

The invention further provides use of a polypeptide according to the invention in a prophylaxis or treatment of a cancer such as a gastro-intestinal cancer or prostate cancer.

Methods of prophylaxis or treating a cancer, such as a gastro-intestinal cancer, or prostate cancer, by administering a protein or peptide according to the invention to a patient, are also provided.

Vaccines comprising nucleic acid and/or polypeptides according to the invention are also provided. The polypeptide may be attached to another carrier peptide such as tetanus toxoid to increase the immunogenicity of the polypeptide.

The polypeptides of the invention may be used to raise antibodies. In order to produce antibodies to tumour-associated antigens procedures may be used to produce polyclonal antiserum (by injecting protein or peptide material into a suitable host) or monoclonal antibodies (raised using hybridoma technology). In addition phage display antibodies may be produced, this offers an alternative procedure to conventional hybridoma methodology. Having raised antibodies which may be of value in detecting tumour antigen in tissues or cells isolated from tissue or blood, their usefulness as therapeutic reagents are likely to be assessed. Antibodies identified for their specific reactivity with tumour antigen may be conjugated either to drugs or to radioisotopes. Upon injection it is anticipated that these antibodies localise at the site of tumour and promote the death of tumour cells through the release of drugs or the conversion of pro-drug to an active metabolite. Alternatively a lethal effect may be delivered by the use of antibodies conjugated to radioisotopes. In the detection of secondary/residual disease, antibody tagged with radioisotope could be used, allowing tumour to be localised and monitored during the course of therapy. Unconjugated antibodies can also be useful in influencing cancer cell growth. For example, the binding of certain antibodies to cell-surface receptors on cancer cells may initiate cell death by, e.g., apoptosis. Therefore the antibodies of this invention are likely to be therapeutically useful in a non-conjugated form.

The term “antibody” includes intact molecules as well as fragments such as Fa, F(ab′)₂ and Fv.

The invention accordingly provides a method of treating a cancer such as gastro-intestinal cancer, or prostate cancer, by the use of one or more antibodies raised against a polypeptide of the invention.

The cancer-associated proteins identified may form targets for therapy.

The invention also provides nucleic acid probes capable of binding sequences of the invention under high stringency conditions. These may have sequences complementary to the sequences of the invention and may be used to detect mutations identified by the inventors. Such probes may be labeled by techniques known in the art, e.g. with radioactive or fluorescent labels.

Preferably the cancer which is detected, assayed for, monitored, treated or targeted for prophylaxis, is a gastric cancer or prostate cancer.

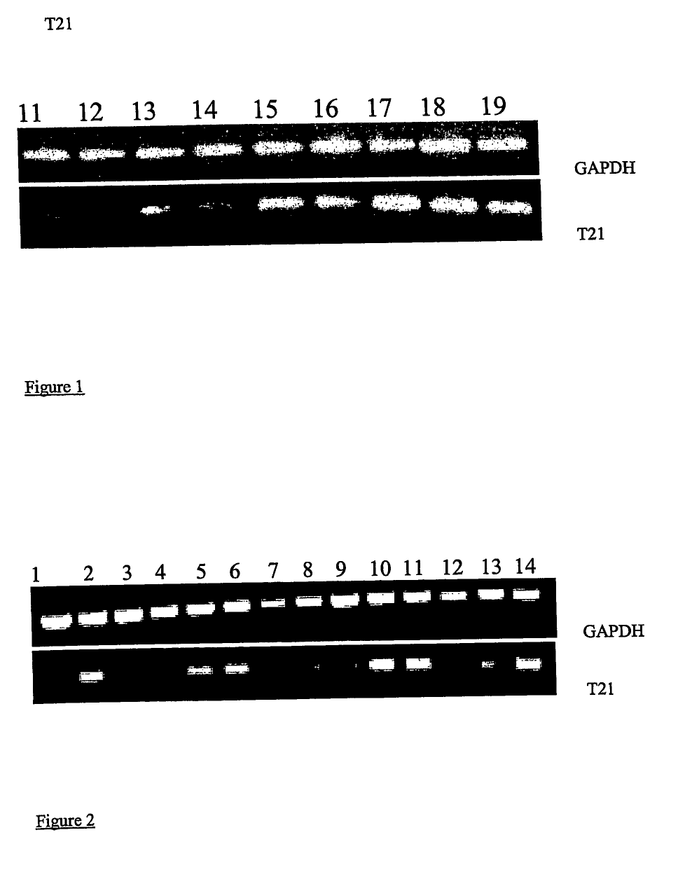

The invention will now be described by reference to the following figures and examples: FIG. 1

Expression of T21 analysed by RT-PCR. T21 expression in normal tissues is restricted to testis and prostate. Lanes: 1, lung; 2, liver; 3, kidney; 4, brain; 5, trachea; 6, heart; 7, gastric tumour; 8, gastric tumour; 9, kidney tumour; 10, kidney tumour; 11, BPH; 12, BPH; 13, BPH; 14, normal prostate; 15, normal testis; 16, normal testis; 17, normal testis; 18, prostate cancer; 19, prostate cancer. In contrast T21 transcripts are detectable with various frequencies in tumour specimens, as shown for selected gastric, kidney and prostate samples. Varying levels of T21 expression are also found in selected BPH samples. The same cDNA samples were tested for GAPDH, as an internal control.

FIG. 2. Expression of T21 analysed by RT-PCR. Lanes: 1, HT29; 2, Jurkat; 3, FM3; 4, LNCap; 5, DU145; 6, PC3; 7, CaP 1; 8, CaP 2; 9, CaP 10; 10, CaP 11; 11, CaP 15; 12, CaP 16; 13, CaP 36; 14, CaP 92/41. T21 transcripts are detectable with various frequencies in tumour cell-lines and prostate cancer samples. No expression of T21 is observed in LNCap, CaP 1 and CaP 16 samples, whilst low levels of expression are observed in HT29, FM3, CaP 2, 10 and 13. High level expression of T21 is observed in Jurkat, DU145, PC3, CaP 2, 10 and 36 samples and T21 is over expressed in CaP 10, 11 and CaP 92/41 when compared to GAPDH. The same cDNA samples were tested for GAPDH, as an internal control.

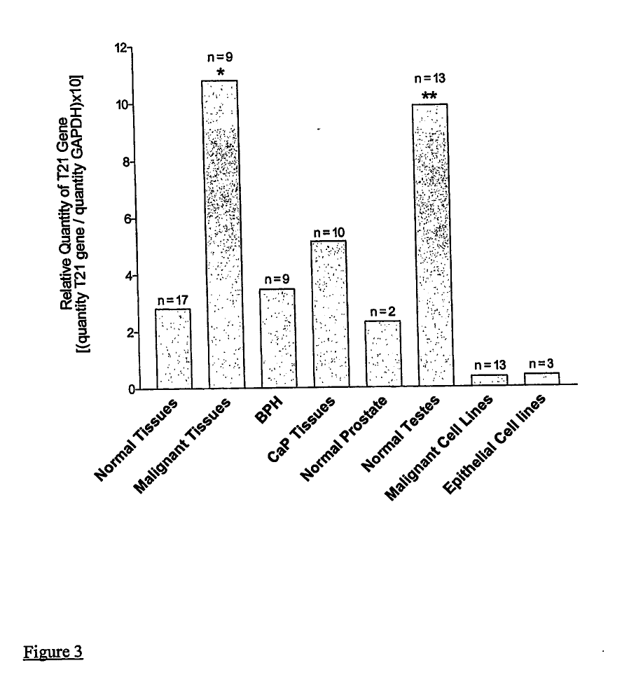

FIG. 3.

Tissue expression analysis of T21 transcripts analysed by RT-Q-PCR. To calculate an arbitrary level of expression of T21 using RT-Q-PCR a standard curve was generated using serial dilutions of testis cDNA as template and assigning the dilutions arbitrary concentration values. T21 gene specific primers were then used in the RT-Q-PCR reactions to generate gene specific product. A panel of normal and malignant samples was run in parallel to the standard curve and arbitrary quantities of T21 expression were calculated from the standard curve. The same cDNA samples were tested for GAPDH, an internal control. These expression levels were then normalised to GAPDH by dividing the quantity of T21 gene specific product by the quantity of GAPDH in the same sample. T21 transcripts are over expressed in malignant tissues (gastric and kidney) (p<0.05) and normal testes (p<0.01) when compared to normal tissues (lung, liver, heart, brain, trachea, kidney, adrenal gland, endometrium, colon, breast, PBMC, tonsil, small intestine, vagina, muscle, placenta and ovary.

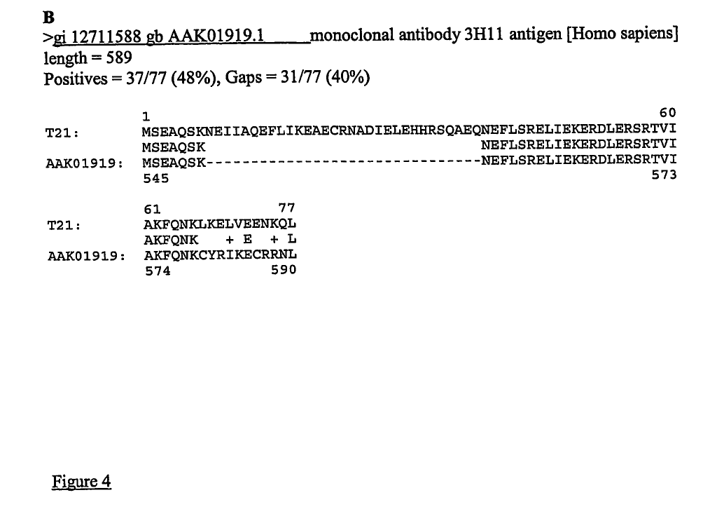

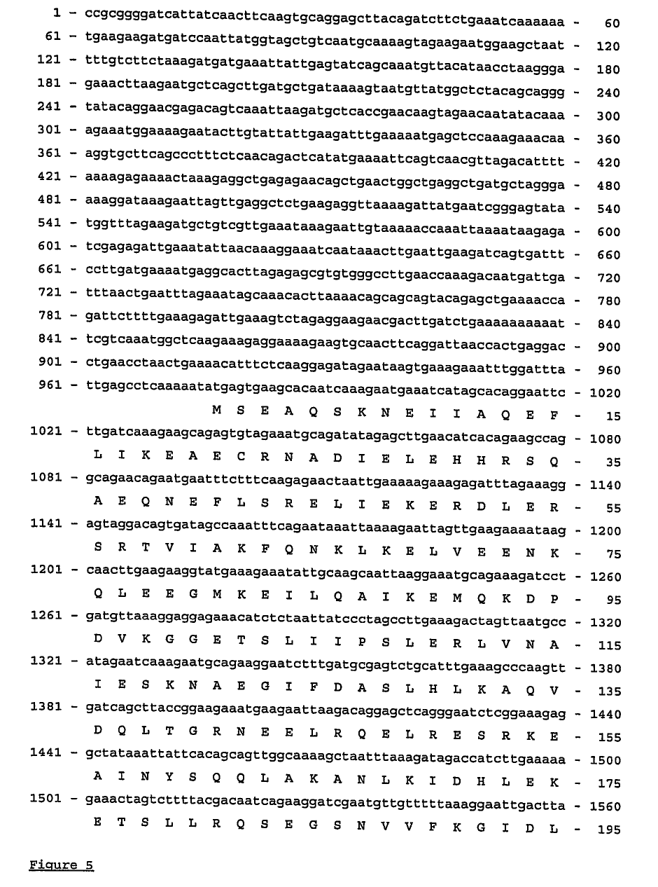

FIG. 4.

Comparison of the T21 sequence against the protein bound by MAb 3H11.

FIG. 5.

The full T21 clone sequence.

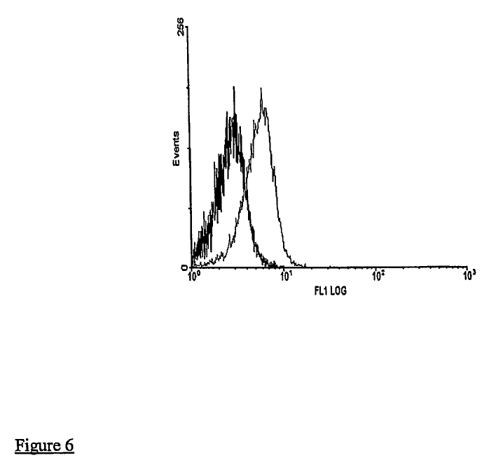

FIG. 6.

Stabilisation of MHC class I with T21:765 peptide on the surface of T2 cells. A typical histogram for T21:765 and DMSO control are presented. The black line histogram shows the stabilisation of MHC class I with the control (DMSO); the red line histogram shows the stabilisation of MHC class I with T21:765 peptide. Data presented are representative of three experiments.

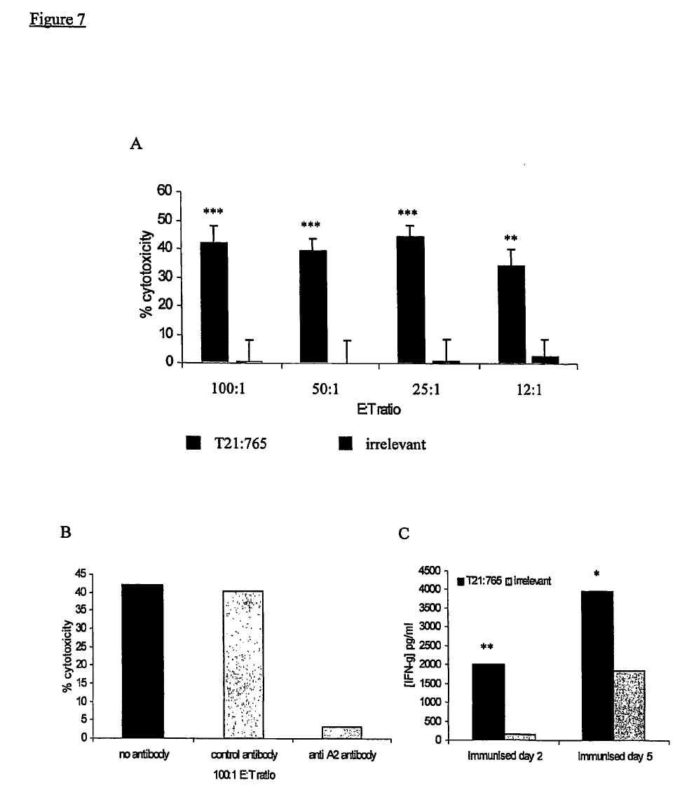

FIG. 7.

Summary graphs of T21:765 immunised HHD II mice (n=9). The graphs are representative of 3 separate experiments involving 3 mice in each experiment.

(A) Mean CTL activity of splenocytes cultured in vitro following immunisation with 100 μg of T21:765 peptide and 140 μg of Hep B class II helper peptide. One week post immunisation the CTL activity of splenocytes cultured for 5 days in vitro was assessed using RMAS-A2 transfected cells pulsed with peptides (T21:765 relevant, gp100 irrelevant).

(B) Effect of HLA-A2.1 blocking antibody on splenocyte cytotoxicity of RMAS-A2 peptide pulsed target cells.

(C) Days 2 and 5 in vitro culture supernatants were collected and assayed for IFN-γ concentration by ELISA and the values were expressed as pg of IFN-γ produced per ml of supernatant. ***p<0.001, **p<0.01 and *p<0.05 (paired Students T test).

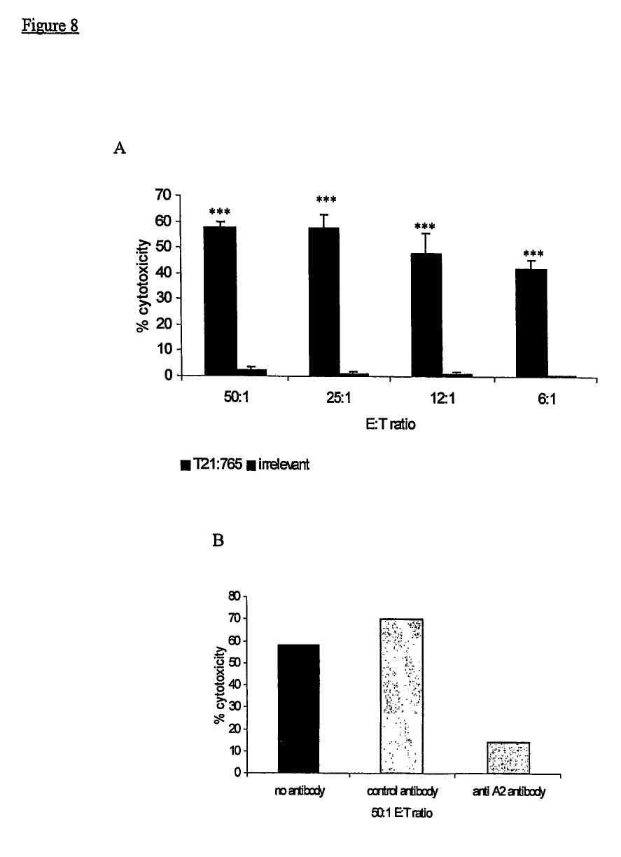

FIG. 8.

Summary graphs of T21:765 immunised HHD II mice (n=5) using the LPS blast method. The graph is representative data from 2 experiments.

(A) Mean CTL activity of splenocytes cultured in vitro following immunisation with 100 μg of T21:765 peptide and 140 μg of Hep B class II helpeer peptide. Two days prior to spleen removal, splenocytes from a naïve mouse were cultured with lipopolysaccharide and dextran sulphate. One week post immunisation the CTL activity of immunised mouse splenocytes cultured for 5 days in vitro with LPS blast naïve splenocytes, pulsed with 100 μg/ml relevant or irrelevant (PAP135) peptide, was assessed using RMAS-A2 transfected cells pulsed with peptides (T21:765 relevant, gp100 irrelevant).

(B) Effect of HLA-A2.1 blocking antibody on splenocyte cytotoxicity. ***p<0.001 (paired Students T test).

Technique Used to Identify Genes Encoding Tumour Antigens (SEREX Technique)

The technique for the expression of cDNA libraries from human testes is described, and was performed according to published methodology (Sahin et.al. Proc Natl. Acad. Sci. 92, 11810-11813, 1995).

SEREX has been used to analyze gene expression in tumour tissues from human melanoma, renal cell cancer, astrocytoma, oesophageal squamous cell carcinoma, colon cancer, lung cancer and Hodgkin's disease. Sequence analysis revealed that several different antigens, including HOM-MEL-40, HOM-HD-397, HOM-RCC-1.14, NY-ESO-1, NY-LU-12, NY-CO-13 and MAGE genes, were expressed in these malignancies, demonstrating that several human tumour types express multiple antigens capable of eliciting an immune response in the autologous host. This represents an alternative and more efficient approach to identify tumour markers, and offers distinct advantages over previously used techniques:
1) the use of fresh tumour specimens to produce the cDNA libraries obviates the need to culture tumour cells in vitro and therefore circumvents artifacts, such as loss of neo-antigen expression and genetic and phenotypic diversity generated by extended culture;
2) the analysis is restricted to antigen-encoding genes expressed by the tumour in vivo;
3) using cDNA expression cloning, the serological analysis (in contrast to autologous typing) is not restricted to cell surface antigens, but covers a more extensive repertoire of cancer-associated proteins (cytosolic, nuclear, membrane, etc.);
4) in contrast to techniques using monoclonal antibodies, SEREX uses poly-specific sera to scrutinise single antigens that are highly enriched in lytic bacterial plaques allowing the efficient molecular identification of antigens following sequencing of the cDNA. Subsequently the tissue-expression spectrum of the antigen can be determined by the analysis of the mRNA expression patterns using, for example, northern blotting and reverse transcription-PCR (RT-PCR), on fresh normal and malignant (autologous and allogeneic) tissues. Likewise, the prevalence of antibody in cohorts of cancer patients and normal controls can be determined.

The T21 sequence can be isolated by the polymerase chain reaction using the following pair of primers:

| | |
|---|---|
| T21 Primer 1: | |
| CAGCTTACCGGAAGAAATGAA | SEQ ID No. 14) |
| T21 Primer 2: | |
| GATGGTGCTATCCCATCAGG | (SEQ ID No. 15) |

under the following reaction conditions:

| Temperature (° C.) | Time (minutes) | Cycles | Comment |
|---|---|---|---|
| 95 | | | Denaturation |
| 95 | | | Denaturation |
| 58 | | | Annealing |
| 72 | | | Extension |
| 95 | | | Denaturation |

The terms ‘denaturation’, ‘annealing’ and ‘extension’ are well-known and understood to the person skilled in the art of PCR and the reader is directed to ‘Figure 10.1 in Principles of Gene Manipulation. An Introduction to Genetic Engineering (5^{th} Edition, 1994). R. W. Old and S. B. Primrose (Publisher: Blackwell Science).

Extraction of Nucleic Acid From Tissue

A prokaryotically expressed cDNA library can be constructed by isolating 10 μg of total RNA from normal testes tissues, treating the total RNA with Calf Intestinal Phosphatase to remove 5′-phosphates from uncapped RNAs, removing the cap structure from full-length mRNA by Tobacco Acid Pyrophosphatase (TAP) and ligating RNA adapters to mRNA molecules containing 5′phosphate. The actual library used was a commercially available λTriplEx2™ Human testes large-insert cDNA library (Product 634220, Clontech, Palo Alto, Calif., USA).

Serological Analysis of Recombinant cDNA Expression Libraries (SEREX)

The SEREX approach allows an unbiased search for an antibody response and the direct molecular definition of immunogenic tumour proteins based on their reactivity with allogeneic patient sera. In this approach, a prokaryotically expressed cDNA library constructed from normal human testes was immunoscreened with absorbed and diluted patients' sera for the detection of tumour antigens that have elicited a high-titer immunoglobulin (Ig) G humoral response. Such a humoral response implies T-cell recognition of the detected antigens by helper T cells. Thus, even though the antigens are initially identified by antibodies, the method reveals tumour products that can then be analysed in the context of cell-mediated immunity. The SEREX approach can then be modified and used to determine the reactivity of identified antigens with panels of human sera including prostate cancer patient sera and normal donor sera.

In this case, the SEREX approach was modified by pooling allogeneic sera from four prostate cancer patients to screen a normal testes cDNA library, rather than a cancer cDNA library, to identify cancer-testes (C-T) antigens.

Inserts were sequenced on a ABI Prism semi-automated sequencer using T7 primers specific for the vector.

Rapid Amplification of the cDNA Ends (RACE)

Sequencing of the clones identified following immunoscreening allows only the cDNA insert sequence to be in attained full. The complete 5′- and 3′-ends of the sequence can then be obtained using a procedure termed rapid amplification of cDNA ends (RACE). The SMART™ RACE cDNA amplification kit (BD Biosciences, Clontech, Palo Alto, Calif., USA) used provides a novel method for performing both 5′- and 3′-RACE. In brief, first-strand cDNA synthesis is performed on high quality testes RNA expressing the gene of interest. The cDNA for 5′-RACE-ready cDNA is synthesised using a modified lock-docking oligo (dT) primer and the SMART II oligo. The 3′-RACE-ready cDNA is synthesised using a traditional reverse transcription procedure but with a special oligo (dT) primer. This primer also has a portion of the SMART sequence at its 5′ end. By incorporating the SMART sequence into both the 5′- and 3 ′-RACE-ready cDNA populations, both PCR reactions can be primed using the universal primer mix (UPM) A, that recognises the SMART sequence, in conjunction with distinct gene-specific primers designed to amplify either in the 5′ or 3′ direction.

Setting Up 5′ and 3′ RACE PCR Reactions

| | 5′-RACE | | 3′-RACE | |
|---|---|---|---|---|
| Component | sample | | sample | |
| RACE-Ready cDNA | 2.5 | μl | 2.5 | μl |
| UPM (10X) | 5 | μl | 5 | μl |
| 5′ RACE gene specific primer (SEQ ID No 4) | 1 | μl | | |
| CCTCTTTCCGAGATTCCCTGAGCTCC | | | | |
| 3′ RACE gene specific primer (SEQ ID No 5) | | | 1 | μl |
| GAACATCACAGAAGCAGGCAGAACA | | | | |
| Master Mix | 41.5 | μl | 41.5 | μl |

Following the primary PCR reaction a ‘nested’ PCR reaction was undertaken using the following nested gene specific primer:

5′ RACE gene specific nested primer:

| | |
|---|---|
| TCACAGAAGCCAGGCAGAACAGAATGA | (SEQ ID No 6) |

3′ RACE gene specific nested primer:

| | |
|---|---|
| GACTTACCTGATGGGATAGCACCATCT | (SEQ ID No 7) |

For T21 5′ RACE PCR the following For T21 3′ RACE PCR the following cycling conditions were used: cycling conditions were used:

| | |
|---|---|
| 5 cycles | 5 cycles |
| 94° C. 15 secs | 94° C. 15 secs |
| 72° C. 3 mins | 72° C. 3 mins |
| 5 cycles | 94° C. 15 secs |
| 94° C. 15 secs | 5 cycles |
| 70° C. 15 secs | 70° C. 15 secs |
| 72° C. 3 mins | 72° C. 3 mins |
| 94° C. 15 secs | 94° C. 15 secs |
| 32 cycles | 33 cycles |
| 68° C. 15 secs | 68° C. 15 secs |
| 72° C. 3 mins | 72° C. 3 mins |

‘Nested’ RACE PCR

Following the primary PCR reaction a ‘nested’ PCR reaction was undertaken using the reaction setup outlined above with the following alterations: 2.5 μl of primary PCR product, 1 μl of nested universal primer and 1 μl of nested gene specific primer:

T21 5′ RACE gene specific nested primer:
TCACAGAAGCCAGGCAGAACAGAATGA
T21 3′ RACE gene specific nested primer:
GACTTACCTGATGGGATAGCACCATCT

The following cycling conditions were The following cycling conditions were used for T21 5′ RACE nested PCR: used for T21 3′ RACE nested PCR:

| | |
|---|---|
| 20 cycles | 20 cycles |
| 94° C. 15 secs | 94° C. 15 secs |
| 68° C. 15 secs | 66° C. 15 secs |
| 72° C. 3 mins | 72° C. 3 mins |

The reactivity of promising positive clones against patient and normal donor serum was determined by immunoscreening the clones against and allogeneic panel of 10 prostate cancer patients' sera and 10 healthy donor patients' sera. The methodology used was the same as the SEREX methodology (described above) with the following modifications: The positive clone (T21) and a negative clone (blue phage) were plated out on a small LB agar plate at a ratio of 1:2 to give a titre of approximately 600 pfu/plate. The steps for detection of false positives, subcloning and retesting were not necessary. Following expression overnight, plaque expression and transferral of the plaques to nitrocellulose membranes, the nitrocellulose membranes were incubated with the individual serum and colour developed as described previously. The results would then be recorded simply as being positive (plaques visible) or negative (no plaques visible).

Quantitiative Real Time Reverse Transcription Polymerase Chain Reaction (RT-Q-PCR)

RT-Q-PCR was used to determine the tissue specificity of T21 expression of SEREX-defined genes in various tissues and cell lines. The indicator dye used was SYBR green.

RT-Q-PCR quantitates the initial amount of template most specifically, sensitively and reproducibly, and is a preferable alternative to semi-quantitative RT-PCR which detects the amount of final amplified product. Real time PCR monitors the fluorescence emitted during the reaction as an indicator of amplicon production during each PCR cycle as opposed to the endpoint detection by conventional PCR methods. The quantitative detection of the amplicon can be measured using a DNA-binding agent called SYBR green (Molecular Probes Inc., Eugene, Oreg., USA). SYBR green is a non-sequence specific fluorescent intercalating agent that only binds to double stranded DNA within the minor groove.

The Mx4000 apparatus measures the fluorescence of each sample at the end of the annealing step, and at the end of each cycle when creating the dissociation curve. After the RT-PCR reaction, the software program plots linear values of fluorescence (dRn) against cycle number. After background adjustment, the Ct value, which is defined as the number of cycles at which the reaction crosses a threshold value, i.e. the fluorescence due to the RT-PCR product exceeds the background level, is calculated for each sample by the software. The software produces a standard curve by measuring the Ct value of each standard and plotting it against the approximate concentrations for the corresponding standard dilution. The expression level of the unknown genes in a given RNA sample are then normalised to the housekeeping gene GAPDH. The normalised expression of each gene is calculated by dividing the Ct value for the unknown gene in a sample by the Ct value for GAPDH in the same sample. Thus, a sample with high level expression of a gene will have a lower Ct value because the gene is more abundant, hence it takes less cycles for the fluorescence to exceed that of background levels. Therefore, when calculating the normalised expression for that gene the Ct value would be lower than a gene that is less abundant. This should be remembered when observing the normalised expression graphs because the lower the Ct value the more abundant the gene is in the sample. Derivation of this fraction is independent of RNA sample concentration, eliminating the requirement to measure RNA concentration accurately.

The RT-Q-PCR reactions were performed in the Mx4000 QPCR system (Stratagene, UK) using SYBR green fluorescent dye (Yin, J. L. et al., 2001. Immuno. Cell Biol. 79(3):213-21). RNA samples were DNase treated in order to remove genomic DNA following standard protocols. Thermocycling for each reaction was done in a final volume of 25 μl containing 1 μl of template (1:10 diluted), or standard, 12.5 μl SYBR green master mix (Qiagen, UK) containing Hot Start®Taq DNA polymerase, reaction buffer, ROX reference dye, SYBR green dye, magnesium chloride and deoxynucleotides, and pre-optimised amounts of gene-specific forward and reverse primers. This was then made up to 25 μl with Qiagen water. In each experiment a minimum of 8 no-template controls should be included to ensure no contamination has occurred and also to indicate the degree of amplification due to primer dimers. Also included were 4 RT-negative (no reverse transcription) samples to ensure that genomic DNA had been completely removed following DNase treatment.

Gene specific primer sequences used in both conventional and real time PCR:

| | | Sequence |
|---|---|---|
| Gene | Primer Sequences (5′ → 3′) | listing reference |
| GAPDH | GGTGAAGGTCGGAGTCAACGGA | SEQ ID No 8 |
| | GAGGGATCTCGCTCCTGGAAGA | SEQ ID No 9 |
| T5 | CGAGCATTCAGGGAACAAGT | SEQ ID No 10 |
| | CCAATGTCAAGAGTGGCAGA | SEQ ID No 11 |
| T17 | CGACCAGGACTCCTACTCCA | SEQ ID No 12 |
| | AGGATCATGGAGGGCTTCTT | SEQ ID No 13 |
| T21 | CAGCTTACCGGAAGAAATGAA | SEQ ID No 14 |
| | GATGGTGCTATCCCATCAGG | SEQ ID No 15 |
| T81 | ACTGGGCGTAAGAAAAGCAAA | SEQ ID No 16 |
| | CATGGGATGTGGAGTTTCTG | SEQ ID No 17 |
| T102 | TGGAGGGCGGTACTTTACAA | SEQ ID No 18 |
| | TGCTTATTTCCCCAGACACC | SEQ ID No 19 |
| T128 | GAGAGAGCGATCAAGAGAAAGG | SEQ ID No 20 |
| | ATCTCTGTGCCGCCTATCAT | SEQ ID No 21 |

In Silico Analysis of T21

The amino acid sequence of T21 was analysed using search programs including PROSITE (accessible at www.expasy.ch/prosite/), PSORT (accessible at http://psort.nibb.ac.jp) and Pfam (accessible at http://www.sanger.ac.uk/cgi-bin/Pfam/nph-search.cgi).

Results

Tissue Specificity

The results of tissue specificity studies are shown in Table 1 and FIGS. 1 to 3. Table 2 shows the reactivity of patients sera with T21.

Immune recognition of identified gene products in prostate cancer patients. To assess the frequency of antibody responses against T21 in sera from normal individuals and prostate cancer patients, 10 prostate cancer patient sera and 10 normal adult sera were screened using a modified immunoscreening procedure. T21 antigen reacted exclusively with allogeneic sera obtained from prostate cancer patients but not with sera from healthy individuals (data not shown).

The antigen is testes specific in normal tissues, but is highly expressed in cancerous tissues.

Sequence

T21 was sequenced and the homology of sequence was compared with homologous sequences in Genbank, the inventors found that clone T21 has sequence identity that was highly restricted as shown in Table 3. The mRNA expression pattern of T21 was tested using both conventional RT-PCR and quantitative RT-PCR methods. T21 gene-specific primers (Table 1: SEQ ID No 14 and SEQ ID No 15) were designed based on the full sequence obtained for this clone

Conventional RT-PCR analysis showed a strong signal for testis (3/3) and a weak signal in normal prostate. All other normal tissues tested were negative (lung, liver, heart, kidney, brain, trachea) (data shown in FIG. 2a).

T21 expression in prostate cancer, BPH and other tumours was examined. Ten prostate cancer samples were tested and 8 out of 10 (80%) were positive by RT-PCR (5 showed strong signals and 3 showed moderate signals; data are shown in FIGS. 2a(ii) and b. Among 3 BPH samples and 4 non-prostate tumour samples tested (2 gastric cancer and 2 kidney cancer), two out of three BPH samples and both gastric samples showed weak to moderate expression of T21, whilst weak expression was observed in both kidney cancer samples tested (FIGS. 2a(i) and (ii)).

The expression of T21 was also examined in malignant cell lines derived from colon cancer (HT29), leukaemia (Jurkat), melanoma (FM3), and prostate cancer (LNCaP, DU145 and PC3). High levels of expression were observed in the leukaemic cell line, and two out of three prostate cancer cell lines (DU145 and PC3), with weak expression observed in the colon cancer cell line (HT29) and melanoma cell line (FM3) (FIG. 2b). The tissue expression pattern of T21 is summarized in Table 2.

The standard curve constructed for the quantitative RT-PCR (Q-PCR) measurement of T21 expression showed good dynamic range with the ability to detect T21 transcripts over 6 orders of magnitude (data not shown). The linear relationship between the cycle threshold (Ct) and relative initial quantity was strong (r²=0.995). The housekeeping gene GAPDH was chosen as the normaliser for T21 making the Q-PCR data comparable with the conventional PCR data. FIG. 3 illustrates the results obtained for Q-PCR analysis on T21 expression in a variety of tissues. The mean expression of T21 was 4-fold higher in malignant tissues (gastric cancer and kidney cancer) than in the normal tissues tested (lung, liver, heart, brain, trachea, kidney, adrenal gland, endometrium, colon, PBMC, breast, small intestine, vagina, muscle and ovary) (p<0.05). The mean value of T21 expression in normal testes was 3.5 times higher than in the same panel of normal tissues (p<0.01). The expression of T21 in the prostate cancer samples tested was 2 times higher than in the normal tissues, however, the difference was not statistically significant.

A comparison of the sequence against the protein bound by MAb 3H11 is shown in FIG. 4.

The sequence of the coding part of T21 is shown in SEQ. ID. No. 2 and SEQ. ID No. 3. The fill sequence is shown in FIG. 5 and SEQ. ID. No. 1.

In Silico Structural Analysis of T21 cDNA.

DNA sequencing of the T21 cDNA insert revealed a 694 bp cDNA sequence. This represented a continuous ORF throughout this sequence, indicating that this sequence is truncated at both 5′ and 3′ ends. Comparison of the 3′ end of the sequence with non redundant entries in the GenBank database revealed significant homology to entry AK025632, however the nucleotide sequence of this entry coded only for a partial protein when translated. An additional entry, BC008641, had homology to AK025632 and this encoded a full protein sequence. The combination of AK025632 and BC008641 extended 1019 bp further in its 3′ sequence than T21 cDNA clone and allowed the definition of the translational termination codon, with a 3′ untranslated region of 201 bp.

However, no definite translation initiation site was identified in the 5′ unique sequence of T21 and in order to obtain the complete the cDNA sequence, 5′ RACE experiments were performed using T21 gene-specific primers and testicular cDNA as a template. The 5′RACE PCR product was cloned into TA TOPO plasmid vector and sequenced. This 5′RACE sequence extended the cDNA sequence 1068 bp further 5′ with the longest ORF starting at the ATG codon at position 326 with an additional 5′ untranslated region of 975 bp.

The resulting T21 cDNA is 2781 bp in length encoding a putative 535 amino acid protein as shown in FIG. 5. The amino acid sequence of T21 was analysed using search programs including PROSITE (accessible at www.expasy.ch/prosite/), PSORT (accessible at http://psort.nibb.ac.jp) and Pfam (accessible at http://www.sanger.ac.uk/cgi-bin/Pfam/nph-search.cgi). These analyses identified:

(i) a N-glycosylation site at amino acid positions 158-161, 430-433 and 466-469;

(ii) cAMP- and a cGMP-dependent protein kinase phosphorylation site at positions 314-317 and 487-490; (iii) casein kinase II phosphorylation sites at positions 6-9, 34-37, 152-155, 209-212, 234-237, 258-261, 269-272, 332-335, 388-391, 390-393, 421-424, 432-435, 468-471, 478-481 and 490-493;

(iv) N-myristoylation sites at positions 99-104, 123-128, 198-203 and 465-470; and

(v) leucine zipper patterns at positions 214-235 and 319-340.

The presence of a bZIP site (DNA binding site followed by a leucine zipper motif) at positions 60-97, 130-165 and 424-453 suggests that this nuclear protein may function as a transcription factor (Jager, D. et al., 2001, Cancer Research 61: 2055-2061). In addition, k-box regions at positions 33-48 and 511-533 are commonly associated with SRF-type transcription factors. Protein kinase C sites at positions 139-141, 452-154, 258-260, 262-264, 269-271, 313-315, 403-405, 432-434, 486-488 and 516-518 suggest that this clone might be involved in signalling pathways (Park, S. et al., 2003 Biochimica and Biophysica Acta 1625: 173-182). Interestingly, the protein has a SPAN-X domain and this family contains human sperm proteins associated with the nucleus and mapped to the X chromosome. SPAN-X proteins are cancer-testis antigens that are highly insoluble, acidic and polymorphic (Westbrook et al, 2001, Biol. Reprod. 64:345-358). PSORT analysis predicted the nuclear localisation of T21 with 60% confidence. This can be confirmed using conventional assays such as DNA binding assays, e.g. gel shift assays.

Chromosomal Localisation and Exon-Intron Organisation of T21.

Comparison of the sequence of T21 with the human genome allowed the assignment of T21 to chromosome 12q21.33. Exon-intron organisation of T21 was defined by comparison of T21 cDNA with genomic sequences. The amino acid coding region of this gene contains a basic framework of 12 structurally distinct exons, with 9 additional exons encoding 5′ untranslated sequence.

Induction of Cytotoxic T Lymphocytes (CTLs) Specific For T21

1. T21-Deprived Peptides Using Predictive Algorithms

SYFPEITHI, an online computer-assisted algorithm, was used to predict potential HLA-A2.1 restricted peptides derived from the amino acid sequence of T21. The amino acid sequence of T21 was submitted to the program and potential HLA-A2 binding peptides were predicted and scored based on their representative MHC binding affinities. Peptides having a high predicted score with this algorithm are considered to be more likely to bind to the allele of interest than peptides with low scores, therefore peptides displaying high scores for the HLA-A2.1 allele were selected and synthetic peptides were produced (Table 4).

2. Capability of T21-Derived Peptides to Stabilise MHC Class I on the Surface of T2 Cells

Each of the peptides were then screened in a standard T2 assay to determine their ability to stabilise MHC class I molecules on the surface of T2 cells. In brief, T2 cells were cultured routinely in suspension and when required, resuspended to 4×10⁶ cells/ml. 40 μl of the cell suspension was added to round bottom 96 well plates to give a final cell number of 1.6×10⁵ cells/well. Test peptide was added to the wells in triplicate at concentrations of 100, 10, 1 μg. As a control, T2 cells were also treated with equivalent concentrations of DMSO. Following overnight incubation, cells were harvested into FACS tubes, washed and labelled with anti HLA-A2.1 monoclonal antibody (primary antibody) and FITC conjugated goat IgG F(ab′)₂ anti mouse Ig (secondary antibody). Cells were then analysed using a FACScan spectroflurometer. (See Table 5 and FIG. 6).

3. Cytotoxicity Assays

Immunisation Procedure

Transgenic HLA-A2.1/Kb C57 or transgenic C57 HHD II HHD2 mice were given a bolus injection of test peptide (100 μg) and and helper Hep B peptide (100 μg) in PBS emulsified in Incomplete Freunds Adjuvant at a 1:1 ratio subcutaneously at the base of tail. The spleens were harvested between 7 and 10 days post final immunisation later for in vitro cytotoxicity assaysanalysis.

In Vitro Generation of CTLs

Spleens were harvested from the treated and naïve mice. Cells were flushed from the spleen with CTL media using a 25 g needle and syringe. The remaining splenic wall was cut and digested with 1 ml of enzyme cocktail (1.6 mg/ml collagenase and 0.1% DNase) in serum free medium (Sigma Aldrich, UK) at 37° C. in 5% CO2 for 1 hour. Following incubation the spleen tissue was disrupted by pipetting, the cells were collected and combined with the flush cells and centrifuged at 1500 rpm for 5 minutes. The cells were counted using white cell counting fluid (0.6% acetic acid in distilled water) and 0.1% trypan blue. The cells were suspended in CTL media and cultured at 2.5×10⁶ cells/well in a 24 well flat bottomed plate for 5 days at 37° C., 5% CO2 in the presence of 10 μgM of the relevant or irrelevant peptide.

Chromium Release Cytotoxity Assay

Target Cells

RMAS cells are a lymphoblastoid cell-line, which exhibit a deficiency in MHC class-I expression on the cell surface despite synthesising normal HLA-A2 heavy chains and β2-microglobulin. The RMAS cells used in this study are transgenic for the HLA-A2.1/Kb class I molecule present in the HHD II transgenic mice.

RMAS A2 cells were routinely cultured and used as targets in the cytotoxicity assay. The cells were pulsed with 10 μg peptide (relevant and irrelevant) overnight prior to the day of the assay and incubated at 370 C with 5% CO2. Following overnight incubation with peptide, cells were harvested into yellow top tubes and centrifuged at 400 g for 3 mins. Cells were resuspended in residual media and labelled with 1.85 MBq chromium51 for 1 hour at 370 C in a water bath. Cells were washed in serum free media and resuspended in 1 ml of CTL media containing a further 1 μg of peptide. The cells were then allowed to rest by incubating in a water bath at 370 C for a further 45 mins. After this rest period, cells were washed with serum free media and spun at 400 g for 3 mins. Cells were resuspended in 1 ml of CTL media and counted. Cells were then resuspended in CTL media to a concentration of 5×104/ml and used as targets in a chromium release cytotoxicity assay.

Effector Cells

On days 2 and 5 of in vitro restimulation supernatants were collected from the wells for use in ELISA tests measuring IL 10 and IFNγ. On day 5 of in vitro restimulation, splenocytes were harvested from the plates, centrifuged at 400 g for 3 minutes and washed in serum free medium. Cells were counted and resuspended in CTL media to a final concentration of (2.5×10⁶) and used as the effector cells. The cells were resuspended in 1 ml of CTL media and counted. Cell number was adjusted to give a final concentration of 5×106/ml using CTL media. The effector and target cells were serially diluted 1:1 in a 96 well round bottom plate to give effector:target ratios in the range of 100:1 to 12:1 using CTL media as the diluent. 100 μl of target cells were then added to each well. The maximum and spontaneous release of chromium from the target cells was also measured in order to calculate the specific cytotoxicity of the target cells by the effector cells. To measure maximum release 100 μl of target cells was added to 80 μl of CTL media and 20 μl 1% SDS, which causes cell lysis and hence release of chromium that has been taken up by the cells. Spontaneous release was determined by adding 100 μl of target cells to 100 μl of CTL media. No effector cells were added to the maximum and spontaneous experiments. The 96 well plates were covered and incubated for 4 hours in a lead box at 37° C., 5% CO2. After the incubation, 50 μl of supernatant was added to Luma plates, taking care not to disturb the cells. The luma plates were then dried over night in a warm air cupboard before the radio activity was measured using a top-count gamma counter. was performed and the specific percentage cytotoxicity was calculated using the following formulae:

Lipopolysaccharide Blast (LPS Blast)

Three days prior to the removal of spleens from immunised mice, a naïve spleen was collected and flushed using T cell media as described above (1 naïve spleen taken for every 3 imumunised spleens). Cells were counted and cultured at 1.5×106 cells/ml in T cell media containing 25 μg/ml lipopolysaccharide and 7 μg/ml dextran sulphate at 370 C, 5% CO2. These cells were then used as the APCs during in vitro restimulation of immunised splenocytes. On the day of isolation of immunised mice splenocytes, LPS treated naïve splenocytes were irradiated (3000 rads) and washed twice. Cells were counted, adjusted to 20×106 cells/ml and incubated with either 100 μg/ml of relevant or irrelevant peptide for at least 1 hour. Following incubation, cells were washed, counted and adjusted to give 1×106 cells/ml; 500 μl of the cell suspension was added to culture plates containing splenocytes from imumunised mice.

When following the LPS blast protocol, minor changes to the treatment of the immunised splenocytes were made. The same protocol as outlined in (c)(ii) above was followed, however immunised splenocytes were plated into culture plates at a concentration of 2.5×10⁶ cells/well (i.e. cells were adjusted to give 5×10⁶ cells/ml; 500 μl added to each well). To these, 5×105 LPS blasts treated with either relevant or irrelevant peptide were added in a volume of 500 μl to give a final volume per well of 1 ml. Supernatants were taken as outlined above for cytokine analysis on day 2 and 5. On day 5, all cells were used as the effectors in a chromium release assay.

Summaries of T21:765 assays are shown in FIGS. 7 and 8.

Conclusions

T21 is an antigen that is testes specific in normal tissue. Cancerous tissue has high levels of expression of this antigen from cancers derived from tissues other than testes. Prostate and gastric cancers have high levels of the antigen expressed, making it a good marker for cancer and a strong candidate for targeting as a therapeutic agent. The immunogenic activity of this antigen is demonstrated by the identification of antibodies in cancer sufferers which assisted in identifying this antigen via the use of the SEREX method.

Initial results indicate that T21 is a transcription factor involved in signalling pathways.

TABLE 1
Tissue specificity of T21 expression investigated using RT-PCR
of SEREX-defined genes in various tissues and cell lines.

| | Expression |
|---|---|
| Tissue | (no. positive/no. tested) |
| Lung | −(0/1) |
| Liver | −(0/1) |
| Kidney | −(0/1) |
| Brain | −(0/1) |
| Trachea | −(0/1) |
| Heart | −(0/1) |
| Gastric tumour | ++(2/2) |
| Kidney tumour | +(2/2) |
| BPH | +(2/3) |
| Normal prostate | +(1/1) |
| Testis | +++(3/3) |
| Prostate cancer | +++(10/14) |
| HT29 | +(1/1) |
| Jurkat | +++(1/1) |
| FM3 | +(1/1) |
| LNCap | −(0/1) |
| DU145 | ++(1/1) |
| PC3 | +++(1/1) |

The level of expression was determined by the intensity of ethidium bromide stained RT-PCR products:
+++, strong amplification;
++, moderate amplification;
+, weakamplification;
−, no amplification

TABLE 2
Immunomic analysis of SEREX identified genes: Reactivity with sera from prostate cancer
patients and normal individuals.

| | Prostate Cancer Patients' Sera | | | | | | | | | | | Normal Individual Sera | | | | | | | | | | |
|---|---|---|---|---|---|---|---|---|---|---|---|---|---|---|---|---|---|---|---|---|---|---|
| Gene | 27 | 29 | 32 | 33 | 34 | 36 | 37 | 38 | 39 | 48 | | 1 | 2 | 3 | 5 | 12 | 13 | 14 | 19 | 20 | 21 | |
| T21 | + | − | − | − | + | − | − | + | + | − | 4/10 | − | − | − | − | − | − | − | − | − | − | 0/10 |

+, sera reactivity observed;
−, no sera reactivity observed.
The above table indicates the sera reactivity of T21 with a panel of 10 prostate cancer patients sera and 10 normal donor sera.
Sera reactivity is only observed to 1 prostate cancer serum. No normal sera showed reactivity with T21

TABLE 3
Clones identified by SEREX and their homologous
sequences in the GenBank database

| | Non redundant Homology | EST Homology |
|---|---|---|
| Clone | (GenBank access no.) | (GenBank access no.) |
| T21 | Hypothetical protein FLJ13615 | Placenta (AU158080) |
| | (XM_037553, XM_017006) | |
| | Hepatoma cells (AK025632) | Hypernephroma cell line |
| | | (BG170100) |
| | Hypothetical protein FLJ13615 | 10 weeks whole embryo |
| | (NM_025114, AK023677) | (AU143956) |
| | 12q BAC RP11-474L23 (AC091516) | Normal epithelium from |
| | | ovary (AI954119) |
| | 12q BAC RP11-66D24 (AC087865) | Osteosarcoma cell line |
| | Mab 3H11 antigen (AF317887) | (BG109374) |
| | Homo sapiens clone from placenta | |
| | choriocarcinoma (BC008641) | |

Homology searching of SEREX-defined clones was determined using NCBI BLAST (http://www.ncbi.nlm.gov/BLAST/).

In brief, the nucleotide sequence of T21 was pasted into blast and subjected to non-redundant and expression sequence tag homology searches. Any clones already submitted to the database with significant homology to T21 are listed in the table above. The letters and numbers in brackets are the blast accession numbers.

TABLE 4
Name, sequence and score of four of the T21 peptides predicted
to bind to HLA-A2.1. Peptides were predicted and scored using the
SYFPEITHI algorithm program available on the World Wide Web
(http://www.uni-tuebingen.de/uni/kxi).

| Peptide name | Sequence* | Score | Seq. ID. No. |
|---|---|---|---|
| T21:765 | YLIHLLQEL (213-221) | 31 | 22 |
| T21:656 | LIIPSLERL (104-112) | 28 | 23 |
| T21:660 | SLERLVNAI (108-116) | 26 | 24 |
| T21:607 | VIAKFQNKL (59-57) | 25 | 25 |

*with position in the sequence of FIG. 5.

TABLE 5
Mean T2 binding ratios calculated for the four T21-derived peptides.
Mean fluorescence (T2 binding ratio) was calculated by dividing the
fluorescence observed for the test peptide by the fluorescence
observed for the control (DMSO).

| | Mean T2 | | |
|---|---|---|---|
| | binding ratio (n = 3) | | |
| Peptide | 1 μg | 10 μg | 100 μg |
| T21:765 | 0.955 | 1.112 | 2.860 |
| T21:660 | 0.918 | 0.942 | 0.893 |
| T21:656 | 0.955 | 0.928 | 0.921 |
| T21:607 | 0.951 | 0.967 | 0.933 |

1. An isolated mammalian nucleic acid molecule selected from the group consisting of:
(a) Nucleic acid molecules encoding a polypeptide comprising the amino acid sequence depicted from SEQ. ID. 3 amino acid number 77 to amino acid 534.
(b) Nucleic acid molecules comprising the nucleotide sequence depicted in FIG. 5 or SEQ. ID. 1 or SEQ.ID. 2.
(c) Nucleic acid molecules, the complementary strand of which specifically hybridises to a nucleic acid molecule in (a) or (b).
(d) Nucleic acid molecules the sequence of which differs from the sequence of the nucleic acid molecule of (C) due to the degeneracy of the genetic code.

2. An isolated nucleic acid molecule according to claim 2, encoding the polypeptide sequence shown in FIG. 5 or SEQ. ID. 3.

3. An isolated nucleic acid molecule which is at least 90% homologous to a nucleic acid sequence as defined in claim 1 or claim 2 and which encodes a polypeptide which is expressed in higher concentrations in cancerous tissue compared to that tissue when in a normal state.

4. An isolated nucleic acid molecule comprising at least 16 nucleic acids capable of specifically hybridising to a sequence within a nucleic acid molecule according to any preceding claim, but not to a nucleic acid sequence encoding the protein bound by MAb 3H12.

5. An isolated nucleic acid molecule according to any preceding claim which encodes a polypeptide which is capable of acting as a transcriptional factor.

6. A vector comprising a nucleic acid molecule according to any preceding claim.

7. A host cell comprising a vector according to claim 6.

8. An isolated polypeptide comprising an amino acid sequence encoded by a nucleic acid molecule according to any preceding claim.

9. An isolated polypeptide according to claim 8 which comprises the amino acid sequence shown in SEQ. ID. No. 3.

10. A fragment or derivative of a polypeptide according to claim 8 or claim 9, which does not contain a sequence of 5 or more amino acids from amino acid residues from 39 to 77 of SEQ. ID. No. 3.

11. A fragment according to claim 10, wherein the fragment comprises a sequence selected from:
(i)

| Peptide name | Sequence |
|---|---|
| T21:765 | YLIHLLQEL |
| T21:656 | LIIPSLERL |
| T21:660 | SLERLVNAI |
| T21:607 | VIAKFQNKL |

(ii) a derivative of one of the peptide sequences (i) having one or more amino acid deletions, additions or modifications, and
(iii) a fragment of the fragment (i) or derivative (ii),
wherein the fragment or derivative has HLA-AZ restricted activity.

12. A isolated nucleic acid molecule which encodes a fragment of a polypeptide according to claim 10 or claim 11.

13. A monoclonal antibody capable of specifically binding to a polypeptide, fragment or derivative according to any one of claims 8 to 10 but not to the protein bound by MAb 3H12.

14. The use of an isolated nucleic acid molecule comprising a sequence according to any one of claims 1 to 5 to detect or monitor cancer.

15. The use of a nucleic acid probe which is capable of specifically hybridising an isolated nucleic acid molecule according to any of claims 1 to 5 or 12.

16. A method of detecting or monitoring cancer comprising the step of detecting or monitoring elevated levels of a nucleic acid molecule comprising a sequence according to claims 1 to 5 or 12 in a sample from a patient.

17. A method of detecting or monitoring cancer comprising the use of a nucleic acid molecule or probe according to claim 14 or claim 15 in combination with a reverse transcription polymerase chain reaction (RT-PCR).

18. A method of detecting or monitoring cancer comprising detecting or monitoring elevated levels of a polypeptide or fragment according to any of claims 10 to 12.

19. A method according to claim 18 comprising the use of an antibody selective for a polypeptide or fragment as defined in any of claims 8 to 11 to detect the polypeptide or fragment.

20. A method according to claim 19 comprising the use of an Enzyme-linked Immunosorbant Assay (ELISA).

21. Use or method according to any one of claims 14 to 20, wherein the cancer is a gastrointestinal cancer, or a prostate cancer.

22. A kit for use with a method according to any one of claims 16 to 21 comprising a nucleic acid, polypeptide, fragment or an antibody as defined in any one of claims 1 to 5, or 9 to 13.

23. A method of prophylaxis or treatment of cancer comprising administering to a patient a pharmaceutically effective amount of nucleic acid molecule comprising a nucleic acid sequence according to any of claims 1 to 5, or 12, or a pharmaceutically effective fragment thereof.

24. A method of prophylaxis or treatment of cancer comprising administering to a patient a pharmaceutically effective amount of a nucleic acid molecule hybridisable under high stringency conditions to a nucleic acid molecule comprising a nucleic acid sequence according to any of claims 1 to 4, or 12, or a pharmaceutically effective fragment thereof.

25. A method of prophylaxis or treatment of cancer comprising administering to a patient a pharmaceutically effective amount of a polypeptide or fragment as defined in any of claims 8 to 10 or a pharmaceutically effective fragment thereof.

26. A method of prophylaxis or treatment of cancer comprising the step of administering to a patient a pharmaceutically effective amount of an antibody according to claim 13.

27. A method according to any one of claims 23 to 26, wherein the cancer is a gastrointestinal cancer.

28. A vaccine comprising a nucleic acid molecule having a nucleic acid sequence as defined in any of claims 1 to 5, or 12, or a pharmaceutically effective fragment thereof and a pharmaceutically acceptable carrier.

29. A vaccine comprising a polypeptide according to any of claims 8 to 11 or a pharmaceutically effective fragment thereof, and a pharmaceutically acceptable carrier.

30. A vaccine according to claim 29 further comprising a carrier molecule for improving the immunogenicity of the polypeptide or fragment.


SEQUENCE LISTING (25 sequences)

SEQ ID NO: 1 - DNA, 2781 bp, Homo sapiens
ccgcggggatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcagccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaagaagtgcaacttcaggattaaccactgaggacctgaacctaactgaaaacatttctcaaggagatagaataagtgaaagaaatttggatttattgagcctcaaaaatatgagtgaagcacaatcaaagaatgaaatcatagcacaggaattcttgatcaaagaagcagagtgtagaaatgcagatatagagcttgaacatcacagaagccaggcagaacagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgccatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgaggtttgatattataagttttatcatacaattatagaataaagaattagttttggtagacattgtattattgttaagtggtttgtctggatctctgaaatatcttattaatatagtgcctatgttttgtgtaataaataaataaaagatttaaatctgaattgtttaaaaggaaaaaaaaaaaaaaaaaaaaaaaaaaaaaaaaaaaaa

SEQ ID NO: 2 - DNA, 1605 bp, Homo sapiens
atgagtgaagcacaatcaaagaatgaaatcatagcacaggaattcttgatcaaagaagcagagtgtagaaatgcagatatagagcttgaacatcacagaagccaggcagaacagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgccatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgaggtttga

SEQ ID NO: 3 - protein, 534 aa, Homo sapiens
MSEAQSKNEIIAQEFLIKEAECRNADIELEHHRSQAEQNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAEV

SEQ ID NO: 4 - DNA, 26 bp, Artificial
  5′ RACE gene specific primer
cctctttccgagattccctgagctcc

SEQ ID NO: 5 - DNA, 25 bp, Artificial
  3′ RACE gene specific primer
gaacatcacagaagcaggcagaaca

SEQ ID NO: 6 - DNA, 27 bp, Artificial
  5′ RACE gene specific nested primer
tcacagaagccaggcagaacagaatga

SEQ ID NO: 7 - DNA, 27 bp, Artificial
  3′ RACE gene specific nested primer
gacttacctgatgggatagcaccatct

SEQ ID NO: 8 - DNA, 22 bp, Artificial
  GAPDH primer 1
ggtgaaggtcggagtcaacgga

SEQ ID NO: 9 - DNA, 22 bp, Artificial
  GAPDH primer 2
gagggatctcgctcctggaaga

SEQ ID NO: 10 - DNA, 20 bp, Artificial
  T5 primer 1
cgagcattcagggaacaagt

SEQ ID NO: 11 - DNA, 20 bp, Artificial
  T5 primer 2
ccaatgtcaagagtggcaga

SEQ ID NO: 12 - DNA, 20 bp, Artificial
  T17 primer 1
cgaccaggactcctactcca

SEQ ID NO: 13 - DNA, 20 bp, Artificial
  T17 primer 2
aggatcatggagggcttctt

SEQ ID NO: 14 - DNA, 21 bp, Artificial
  T21 primer 1
cagcttaccggaagaaatgaa

SEQ ID NO: 15 - DNA, 20 bp, Artificial
  T21 primer 2
gatggtgctatcccatcagg

SEQ ID NO: 16 - DNA, 21 bp, Artificial
  T81 primer 1
actgggcgtaagaaaagcaaa

SEQ ID NO: 17 - DNA, 20 bp, Artificial
  T81 primer 2
catgggatgtggagtttctg

SEQ ID NO: 18 - DNA, 20 bp, Artificial
  T102 primer 1
tggagggcggtactttacaa

SEQ ID NO: 19 - DNA, 20 bp, Artificial
  T102 primer 2
tgcttatttccccagacacc

SEQ ID NO: 20 - DNA, 22 bp, Artificial
  T128 primer 1
gagagagcgatcaagagaaagg

SEQ ID NO: 21 - DNA, 20 bp, Artificial
  T128 primer 2
atctctgtgccgcctatcat

SEQ ID NO: 22 - protein, 9 aa, Homo sapiens
YLIHLLQEL

SEQ ID NO: 23 - protein, 9 aa, Homo sapiens
LIIPSLERL

SEQ ID NO: 24 - protein, 9 aa, Homo sapiens
SLERLVNAI

SEQ ID NO: 25 - protein, 9 aa, Homo sapiens
VIAKFQNKL